(19) United States
(12) Patent Application Publication
(10) Pub. No.: US 2012/0035203 A1
(43) Pub. Date: Feb. 9, 2012

(54) BUTACLAMOL FOR THE TREATMENT OF AMYOTROPHIC LATERAL SCLEROSIS

(75) Inventors: Donald R. Kirsch, Bedford, MA (US); Radhia Benmohamed, Tewksbury, MA (US)

(73) Assignee: CAMBRIA PHARMACEUTICALS, INC., Cambridge, MA (US)

(21) Appl. No.: 13/265,008

(22) PCT Filed: Apr. 27, 2010
(86) PCT No.: PCT/US10/32561

§ 371 Date: Oct. 18, 2011

Related U.S. Application Data
(60) Provisional application No. 61/173,146, filed on Apr. 27, 2009.

(51) Int. Cl. A61K 31/4375; A61P 25/00

(52) U.S. Cl. 514/280

(57) ABSTRACT

The present invention relates to the use of butaclamol and pharmaceutical compositions thereof for treating subjects with amyotrophic lateral sclerosis (ALS) and other neurodegenerative diseases.

### CROSS-REFERENCE TO RELATED APPLICATIONS

This application claims priority to U.S. provisional application Ser. No. 61/173,146, filed Apr. 27, 2009, the entirety of which is hereby incorporated herein by reference.

### BACKGROUND OF THE INVENTION

Amyotrophic lateral sclerosis (ALS) is a fatal neurodegenerative disorder caused by motor neuron death (Rowland et al., N. Engl. J. Med., 2001, 334, 1688-1700) and characterized in part by the presence of abnormal aggregates of insoluble protein in selectively vulnerable populations of neurons and glia. ALS, an orphan disease, is estimated to afflict about 87,000 people worldwide, but its prevalence would be much higher were it not for the fact that ALS patients survive for only 3 to 5 years on average after diagnosis. Approximately 10% of ALS cases are familial, with the rest of the cases being sporadic (Rowland et al., N. Engl. J. Med., 2001, 334, 1688-1700). Approximately 20% of the cases of familial ALS are caused by inherited mutations in the protein Cu/Zn superoxide dismutase (SOD1) (Rosen et al., Nature, 1993, 362, 59-62). Rodent models in mutant SOD1 are often used as a disease model because of its phenotypic and pathologic resemblance to sporadic and familial human ALS (Dal Canto et al., Brain Res., 1995, 676, 25-40; Wong, et al. Neuron, 1995, 14, 1105-1116; Bruijin et al., Science, 1998, 281, 1851-1854; Bruijn et al., Neuron, 1997, 18, 327-338; Wang et al., Hum. Mol. Genet., 2003, 12, 2753-2764; Wang et al., Neurobiol. Dis., 2002, 10, 128-138; Jonsson et al., Brain, 2004, 127, 73-88).

The causes of sporadic ALS remain unknown, and the clinical courses are variable, suggesting that multiple factors are involved. Different hypotheses have been proposed, such as glutamate-mediated excitotoxicity, impaired mitochondrial function, oxidative stress, and aberrant protein aggregation (Dib et al., Drugs, 2003, 63, 289-310; Strong et al., Pharmacology & Therapeutics, 2003, 98, 379-414; Kunst et al., Am, J. Hum. Genet., 2004, 75, 933-947; Bruijn et al., Annu. Rev. Neurosci, 2004, 27, 723-749; Dibernardo et al., Biochimica et Biophysica Acta, 2006, 1762, 1139-1149). Riluzole, which decreases glutamate excitotoxicity, is the only FDA approved ALS drug (Jimonet et al., J. Med. Chem., 1999, 42, 2828-2843.). However, it can only extend median survival life for 2 to 3 months, suggesting mechanisms other than glutamate-mediated excitotoxicity should be considered during ALS drug development (Miller et al., ALS and Other Motor Neuron Disorders, 2003, 4, 191-206; Taylor et al., Neurology 2006, 67, 20-27).

### SUMMARY OF THE INVENTION

The present invention encompasses the recognition that there exists a need for methods for treating patients with amyotrophic lateral sclerosis (ALS) or other neurodegenerative diseases characterized by the presence of aberrant protein aggregates.

The present invention relates to the use of butaclamol to treat neurodegenerative diseases characterized by abberrant protein aggregates. Among other things, the present invention provides methods of treating amyotrophic lateral sclerosis (ALS) with butaclamol. Without wishing to be bound by any particular theory, butaclamol may be useful in the treatment of (ALS) or other neurodegenerative diseases where abnormal protein aggregation has been implicated, as it may prevent the aggregation of protein in a cell or limit the toxicity of such aggregates.

In one aspect, the invention provides methods of treating a subject suffering from or susceptible to a neurodegenerative disease, disorder or condition (e.g., ALS) with butaclamol. In certain embodiments, the subject is an adult human.

In some embodiments, the present invention provides methods of inhibiting or reversing abnormal protein aggregation (e.g., SOD1 protein aggregates) using butaclamol. Inhibiting or reversing abnormal protein aggregation may occur in vivo (e.g., in a subject as described herein) or in vitro (e.g., in a cell).

In some embodiments, the invention provides methods of protecting cells from the cytotoxic effects of aggregated protein (e.g., SOD1) using butaclamol. Protection of cells may occur in vivo (e.g., in a subject as described herein) or in vitro (e.g., in a cell).

In some embodiments, the invention provides methods of modulating proteasome activity in vivo (e.g., in a subject as described herein) or in vitro (e.g., in a cell) using an inventive compound. In certain embodiments, the cells are mammalian cells.

All publications and patent documents cited in this application are incorporated herein by reference in their entirety.

### DEFINITIONS

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, use of the term “about” in reference to dosages means±5 mg/kg/day.

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, 10, 15, or more amino acids are required to be characteristic of a protein. In general, a characteristic portion is one that, in addition to the sequence identity specified above, shares at least one functional characteristic with the relevant intact protein.

Characteristic sequence: A “characteristic sequence” is a sequence that is found in all members of a family of polypeptides or nucleic acids, and therefore can be used by those of ordinary skill in the art to define members of the family.

Intraperitoneal: The phrases “intraperitoneal administration” and “administered intraperitonealy” as used herein have their art-understood meaning referring to administration of a compound or composition into the peritoneum of a subject.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Patient: As used herein, the term “patient”, “subject”, or “test subject” refers to any organism to which butaclamol is administered in accordance with the present invention e.g., for experimental, diagnostic, prophylactic, and/or therapeutic purposes. Typical subjects include animals (e.g., mammals such as mice, rats, rabbits, non-human primates, and humans; insects; worms; etc.). In some embodiments, a subject may be suffering from, and/or susceptible to a disease, disorder, and/or condition (e.g., a neurodegenerative disease, a disease, disorder or condition associated with protein aggregation, ALS, etc.).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Prodrug: A general, a “prodrug”, as that term is used herein and as is understood in the art, is an entity that, when administered to an organism, is metabolized in the body to deliver a therapeutic agent of interest. Various forms of “prodrugs” are known in the art. For examples of such prodrug derivatives, see:
a) Design of Prodrugs, edited by H. Bundgaard, (Elsevier, 1985) and Methods in Enzymology, 42:309-396, edited by K. Widder, et al. (Academic Press, 1985);
b) A Textbook of Drug Design and Development, edited by Krogsgaard-Larsen;
c) Bundgaard, Chapter 5 “Design and Application of Prodrugs”, by H. Bundgaard, p. 113-191 (1991);
d) Bundgaard, Advanced Drug Delivery Reviews, 8:1-38 (1992);
e) Bundgaard, et al., Journal of Pharmaceutical Sciences, 77:285 (1988); and
f) Kakeya, et al., Chem. Pharm. Bull., 32:692 (1984).

The methods and structures described herein relating to butaclamol compounds also apply to pharmaceutically acceptable salts thereof.

Protein: As used herein, the term “protein” refers to a polypeptide (i.e., a string of at least two amino acids linked to one another by peptide bonds). In some embodiments, proteins include only naturally-occurring amino acids. In some embodiments, proteins include one or more non-naturally-occurring amino acids (e.g., moieties that form one or more peptide bonds with adjacent amino acids). In some embodiments, one or more residues in a protein chain contains a non-amino-acid moiety (e.g., a glycan, etc). In some embodiments, a protein includes more than one polypeptide chain, for example linked by one or more disulfide bonds or associated by other means. In some embodiments, proteins contain L-amino acids, D-amino acids, or both; in some embodiments, proteins contain one or more amino acid modifications or analogs known in the art. Useful modifications include, e.g., terminal acetylation, amidation, methylation, etc. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Stereochemically isomeric forms: The phrase “stereochemically isomeric forms”, as used herein, refers to different compounds made up of the same atoms bonded by the same sequence of bonds but having different three-dimensional structures which are not interchangeable. In some embodiments of the invention, chemical compositions may be provided as pure preparations of individual stereochemically isomeric forms of a compound; in some embodiments, chemical compositions may be provided that are or include mixtures of two or more stereochemically isomeric forms of the compound. In certain embodiments, such mixtures contain equal amounts of different stereochemically isomeric forms; in certain embodiments, such mixtures contain different amounts of at least two different stereochemically isomeric forms. In some embodiments, a chemical composition may contain all diastereomers and/or enantiomers of the compound. In some embodiments, a chemical composition may contain less than all diastereomers and/or enantiomers of a compound. Unless otherwise indicated, the present invention encompasses all stereochemically isomeric forms of relevant compounds, whether in pure form or in admixture with one another. If a particular enantiomer of a compound of the present invention is desired, it may be prepared, for example, by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, diastereomeric salts are formed with an appropriate optically-active acid, and resolved, for example, by fractional crystallization.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Tautomeric forms: The phrase “tautomeric forms”, as used herein, is used to describe different isomeric forms of organic compounds that are capable of facile interconversion. Tautomers may be characterized by the formal migration of a hydrogen atom or proton, accompanied by a switch of a single bond and adjacent double bond. In some embodiments, tautomers may result from prototropic tautomerism (i.e., the relocation of a proton). In some embodiments, tautomers may result from valence tautomerism (i.e., the rapid reorganization of bonding electrons). All such tautomeric forms are intended to be included within the scope of the present invention. In some embodiments, tautomeric forms of a compound exist in mobile equilibrium with each other, so that attempts to prepare the separate substances results in the formation of a mixture. In some embodiments, tautomeric forms of a compound are separable and isolatable compounds. In some embodiments of the invention, chemical compositions may be provided that are or include pure preparations of a single tautomeric form of a compound. In some embodiments of the invention, chemical compositions may be provided as mixtures of two or more tautomeric forms of a compound. In certain embodiments, such mixtures contain equal amounts of different tautomeric forms; in certain embodiments, such mixtures contain different amounts of at least two different tautomeric forms of a compound. In some embodiments of the invention, chemical compositions may contain all tautomeric forms of a compound. In some embodiments of the invention, chemical compositions may contain less than all tautomeric forms of a compound. In some embodiments of the invention, chemical compositions may contain one or more tautomeric forms of a compound in amounts that vary over time as a result of interconversion. Unless otherwise indicated, the present invention encompasses all tautomeric forms of relevant compounds, whether in pure form or in admixture with one another.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

For purposes of this invention, the chemical elements are identified in accordance with the Periodic Table of the Elements, CAS version, Handbook of Chemistry and Physics, 67th Ed., 1986-87, inside cover.

### BRIEF DESCRIPTION OF THE DRAWING

FIG. 1. Proteasome inhibition is selectively toxic to PC12 cells expressing mutant G93A SOD1. Cell survival was determined using the viability stain calcein-AM. At 24 hr, treatment with 100 nM MG132 shows no toxicity against any of the cell lines although aggregates are seen in G85R SOD1 and G93A SOD1 cell lines (panel A). Treatment with 100 nM MG132 is selectively toxic to the G93A SOD1 cell line after 48 hours (panel B). Washing of the cells at 24 hours to remove the compound does not reverse toxicity to the G93A SOD1 cell line suggesting that an irreversible toxic event, potentially related the aggregation of G93A SOD1, has been triggered prior to wash out (panel C).

FIG. 2. Radicicol protects PC12 cells expressing mutant G93A SOD1 from the toxic effects of proteasome inhibitor MG132.

FIG. 3. Mutant but not wild type SOD1 aggregates in cells treated with the proteasome inhibitor MG132. Fluorescence micrographs of PC12 cells expressing YFP tagged wild type (WT), G93A mutant (G93A), and G85R mutant (G85R) SOD1 proteins. The micrographs show the effects of treating cells with 200 nM MG132 for 24 h (untreated cells are shown in insets on left). The wild type SOD1 expressing cells are unaffected while cells expressing mutant SOD1 show large perinuclear aggregates.

FIG. 4. Radicicol decreases mutant SOD1 aggregation induced by proteasome inhibitor MG132. Fluorescence micrographs of PC12 cells expressing YFP tagged G93A SOD1 (left) or G85R SOD1 (right) proteins. The cells were treated with 200 nM MG132 to induce protein aggregation, or co-treated with MG132 and radicicol for 24 h. Without radicicol treatment, cells show large perinuclear aggregates. The aggregates are reduced in radicicol-treated cells. While the behavior of the two cell lines is generally similar, G85R SOD1 cells show ‘brighter’ aggregates and more contrast between the aggregates and the cytoplasm.

FIG. 5. Compound microscope fluorescence micrographs of the same G85R SOD1 cells using GFP filter set to image the SOD1-YFP aggregates (above) and the TRITC filter set to image the cells with the Image-iT WGA plasma membrane dye (below).

FIG. 6. Neuroleptic Agent Screening Results (Table 1). Ten neuroleptic therapeutics, respresenting both the ‘typical’ and ‘atypical’ compound types, were screened.

FIG. 7. Comparison of Selected Screening Actives (Table 2). Represents a side by side comparison of the effects of radicicol and butaclamol.

FIG. 8. Dose response curves for (+) and (−) butaclamol diastereomers in the protection assay.

FIG. 9. Open Field Analysis in Butaclamol-Treated G93A mice. Open Field analysis of Wild Type littermate control mice, untreated G93A mice and dose-response using 0.01 mg/kg, 0.1 mg/kg, and 1 mg/kg butaclamol per day in G93A transgenic mice for distance traveled (A), resting time (B), Ambulatory counts (C), and ambulatory time (D). There is a significant difference between WT and untreated G93A mice, with optimal dosing at the 0.1 mg/kg dose significantly different than untreated G93A mice through 105 days and comparable (not significantly different) to WT littermate control mice.

FIG. 10: Kaplan-Meier Survival Curve of Dose Response Using Butaclamol in G93A mice.

FIG. 11. Neuroprotective effects of butaclamol (0.1 mg/kg) in the lumbar spinal cord in G93A transgenic ALS mice. Nissl staining of the lumbar spinal cord from (A) wild-type mice, (B) G93A mice treated with butaclamol, and (C) untreated G93A mice shows a marked gross atrophy in untreated G93A mice. Treatment with butaclamol significantly improved the gross neuropathological changes, as shown in FIGS. 3B and E. Bar in A=200 mm and is the same for B and C. High magnification photomicrographs of Nissl staining in the ventral horn from the same sections in A, B, and C of the lumbar spinal cord from (D) wild-type mice, (E) G93A mice treated with butaclamol, and (F) untreated G93A mice demonstrates ventral neuron loss and atrophy in untreated G93A mice. The treatment with butaclamol markedly improves these neuropathological changes. Bar in D=50 pm and is the same for E and F.

FIG. 12. Glial fibrillary acidic protein (GFAP) expression within the lumbar spinal cord in wild-type mice reveals low levels of reactive astrogliosis (A). In contrast, there is marked astrogliosis, as indicated by GFAP immunostaining, in the lumbar spinal cord from untreated G93A mice (C). Treatment with butaclamol markedly attenuates reactive astrogliosis (B).

### DETAILED DESCRIPTION OF CERTAIN EMBODIMENTS OF THE INVENTION

### Butaclamol

Butaclamol is a type of benzocycloheptapyridoisoquinolinol currently in clinical use for the treatment of schizophrenia. The structure of butaclamol contains a semi-rigid phenethylamine pharmacophore. Butaclamol has proven to be a potent dopamine antagonist and is often used as a reference compound in competitive screens for new antipsychotic agents (Kukla et al., J. Med. Chem. 1979, 22(4), 401-406). Butaclamol exists as optical isomers, with virtually all of the dopamine-blocking activity residing in the (+) isomer. Indeed, for both [3H] dopamine and [3H] haloperidol binding sites, (+) butaclamol is about one hundred and one thousand times more potent, respectively, than the clinically inactive (−) isomer (Shershow, Schizophrenia: Science and Practice, 1978, Harvard University Press). For example, behavioral studies done on rats with unilateral lesions in the substantia nigra have shown that administration of 0.1 to 0.3 mg/kg of the (+) enantiomer abolishes amphetamine-induced stereotyped and rotational behavior, whereas the (−) enantiomer is devoid of behavioral activity even at 100 to 500 times larger than those of the (+) enantiomer. Likewise, while (+) butaclamol antagonized epinephrine-induced mortality at high doses, (−) butaclamol were devoid of any such activity (Voith et al., Can. J. Physiol. Pharmacol. 1976, 54(4), 551-560). In some embodiments of the present invention, compositions are utilized that contain and or deliver (+) butaclamol. In some embodiments, substantially all of the butaclamol so contained and/or delivered is (+) butaclamol. In some embodiments, at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more of the butaclamol so contained and/or delivered is (+) butaclamol.

Butaclamol may exist in several different conformations in solution. The preferred conformations and rates of interconversion of conformers are typically dependent on a variety of factors (e.g., solvent, temperature, free base or salt form of butaclamol, etc.). For example, the HCl salt of butaclamol can exist as an equilibrium mixture of two conformations that differ in their stereochemistry about the ring junction containing the nitrogen atom. In DMSO, the trans form of butaclamol HCl has a relative population of about 80%; in chloroform, the trans form of butaclamol HCl is the only form observed. The free base of butaclamol, in contrast to the HCl salt, interconverts much more readily, with cis/trans conformors observable only at lower temperatures (Casarotto et al., J. Med. Chem. 1991, 34(7), 2043-2049). Conformational energy calculations performed on butaclamol suggest that a trans conformer is the biologically active form that gives rise to the neuroleptic activity of this compound under physiological conditions (Froimowitz et al., Mol. Pharm. 1983, 24(2), 243-250).

Butaclamol compositions used to treat schizophrenia typically contain butaclamol in the form of the free base or in the form of the HCl salt. Common acid addition salts are typically prepared by reacting the base form of butaclamol with the appropriate acid in an organic solvent such as, for instance, ether or an ethanol-ether mixture. These salts, when administered for neuroleptic purposes, often may possess comparable pharmacologic activities as the corresponding bases. For many purposes it is preferable to administer the salt form of butaclamol rather than the free base. Among the acid addition salts suitable for this purpose are salts such as the sulfate, phosphate, lactate, tartrate, maleate, citrate, and hydrochloride (Bruderlein et al., U.S. Pat. No. 3,657,250).

As mentioned above, butaclamol has been extensively evaluated in the treatment of chronic schizophrenic patients. In animals, this compound exhibited a profile suggesting potential antipsychotic effects with potential adverse effects, such as extrapyramidal and adrenergic effects, occurring at doses substantially above the therapeutic range. Phase I studies in healthy volunteers showed that the limiting effects in single and repeated doses up to 32 mg/day were sedation and extrapyramidal effects. Studies on chronic schizophrenic males and females between the ages of 21 and 65 demonstrated that when administered long-term at a dosage of 50 mg/day, butaclamol is indeed an active neuroleptic agent. However, in order to minimize the extrapyramidal effects observed at 50 mg/day, doses in the range of 5-20 mg/day were suggested for initial treatment, and doses as low as 5-10 mg/day were posited as reasonable maintenance doses (Clark et al., J. Clin. Pharmacol. 1977, 17, 529).

### Neurodegenerative Diseases Treated in Accordance With the Present Invention

Imbalances in protein homeostasis are often associated with protein misfolding and/or protein conformational changes that lead to protein aggregation and formation of protein inclusion bodies. Many neurodegenerative diseases, including the polyglutamine (polyQ)-repeat diseases, Alzheimer's disease, Parkinson's disease, prion diseases, frontotemporal lobar degeneration, and ALS, are characterized by the appearance of damaged and aggregated proteins, including huntingtin, polyQ proteins, amyloid A prion (PrP and Sup35) fibrils, and mutant SOD1 (Taylor et al., Science. 2002, 296(5575), 1991-5; Ross, C. A., Neuron. 1997, 19(6), 1147-50; Perutz, M. F., Brain Res Bull. 1999, 50(5-6), 467; and Kopito et al., Nat Cell Bio. 2000, 2(11), E207-9). The fact that such diverse proteins form aggregates in patients with distinct neurological diseases suggests that a common molecular etiology may contribute to the neuropathology in these diseases and that, perhaps, protein misfolding and the subsequent appearance of protein aggregates are early events that play a role in neuronal toxicity in multiple human neurological diseases (Orr, H. T., Genes Dev. 2001, 15(8), 925-32; Ikeda et al., Nat. Genet. 1996, 13(2), 196-202; DiFiglia et al., Science. 1997, 277(5334), 1990-3; Davies et al., Cell. 1997, 90(3), 537-48; Koo et al., Proc Nall Acad Sci USA. 1999, 96(18), 9989-90).

One model for the molecular basis of these neurodegenerative diseases is that insoluble protein aggregates associate and interfere with the activity of other critical soluble cellular proteins, and that loss of function of these diverse proteins has serious negative consequences on cellular function. The affected proteins may include ubiquitin, components of the proteasome, components of the cytoskeleton, transcription factors (TBP (TATA binding protein), EYA (Eyes Absent protein), CBP (CREB binding protein), and molecular chaperones Hsc-70, Hsp-70, Hdj-1, and Hdj-2 (Davies et al., Cell. 1997, 90(3), 537-48; Ross, C. A., Neuron. 2002, 35(5), 819-22; Cummings et al., Nat. Genet. 1998, 19(2) 148-54; Perez et al., J. Cell Biol. 1998, 143(6), 1457-70; Kazantsev et al., Proc Nall Acad Sci USA. 1999, 96(20), 11404-9; Jana et al., Hum Mol. Genet. 2001, 10(10), 1049-59; Nucifora et al., Science. 2001, 291(5512) 2423-8; and Suhr et al., J. Cell Biol. 2001, 153(2), 283-94). Recent studies showed that TBP and CBP are irreversibly sequestered in polyQ/huntington aggregates, while the chaperone Hsp70 is transiently associated with the surface (Chai et al., Proc Natl Acad Sci USA. 2002, 99(14), 9310-5; Kim et al., Nat Cell Biol. 2002, 4(10), 826-31). Sequestration of CBP into polyglutamine aggregates is linked directly with loss of cellular function in neuronal cells, and overexpression of CBP suppressed polyQ toxicity (Nucifora et al., Science. 2001, 291(5512) 2423-8). Furthermore, expression of polyglutamine proteins in C. elegans causes other metastable proteins to lose function. Thus, a single aggregation-prone protein may be able to destabilize protein homeostasis in otherwise normal cells (Gidalevitz et al., Science 2006, 311(5766) 1471-1474). These studies indicate that the sequestration of essential soluble cellular proteins in insoluble protein aggregates could play a significant role in the neuropathology and neurotoxicity in ALS and related diseases.

It is also possible that the cellular mechanism(s) that remove misfolded or damaged proteins (Morimoto, R. I., Cell. 2002, 110(3), 281-4; Horwich et al., Cell. 1997, 89(4), 499-510; and Nollen et al., J Cell Sci. 2002, 115(Pt 14) 2809-16) are overwhelmed in neurodegenerative diseases due to the presence of abundant protein aggregates. The activity of molecular chaperones is one of the most important mechanisms to prevent and/or rescue protein misfolding and aggregation. Molecular chaperones are a large and diverse protein family which includes Hsp104, Hsp90, Hsp70, dnaJ (Hsp40), immunophilins (Cyp40, FKBP), Hsp60 (chaperonins), the small heat shock proteins, and components of the steroid aporeceptor complex (p23, Hip, Hop, Bag1) (Gething, M. J., Nature. 1997, 388(6640) 329, 331; Bakau, b., Amsterdam: Harwood Academic Publishers. 1999, 690). Molecular chaperones ensure proper protein folding by preventing hydrophobic surfaces from interacting with each other, by enhancing protein refolding, and when necessary, by stimulating protein degradation to remove misfolded proteins that tend to aggregate (Horwich et al., Cell. 1997, 89(4), 499-510; Bakau, b., Amsterdam: Harwood Academic Publishers. 1999, 690; Schroder et al., Embo J. 1993, 12(11), 4137-44; Parsell et al., Nature 1994, 372(6505), 475-8; Hartl, F. U., Nature. 1996, 381(6583) 571-9; and Morimoto et al., Nat. Biotechnol. 1998, 16(9), 833-8). Accordingly, overexpression of molecular chaperones can suppress the toxicity of mutant huntingtin, α-synuclein, and SOD1 (Sakahira at al., Proc. Natl. Acad. Sci. USA. 2002, 99 Suppl. 4, 6412-8; Stenoien et al., Hum. Mo.l Genet. 1999, 8(5), 731-41; Warrick et al., Nat. Genet. 1999, 23(4), 425-8; Carmichael et al., Proc. Natl. Acad. Sci. USA. 2000, 97(17), 9701-5; Takeuchi et al., Brain Res. 2002, 949(1-2), 11-22; Auluck et al., Science 2002, 295(5556), 865-8; and Bailey et al., Hum. Mol. Genet. 2002, 11(5), 515-23). Recently, non-chaperone proteins were identified that also suppress toxicity associated with protein aggregation (Kazemi-Esfarjani et al., Science 2000, 287(5459), 1837-40; and Kazemi-Esfarjani et al., Hum. Mol. Genet. 2002, 11(21), 2657-72).

The chaperone system is a highly appealing therapeutic target, because multiple small molecular weight modulators of chaperone activity have already been identified, two of which are active in a mouse model of ALS (Westerheide et al., J. Biol. Chem. 2005, 280(39), 33097-100; Kieran et al., Nat. Med. 2004, 10(4), 402-5; and Traynor et al., Neurology. 2006, 67(1), 20-7). Accordingly, recent analyses identified protein folding/misfolding and protein aggregation as a relevant therapeutic target for neurodegenerative diseases (Pasinelli et el., Nat. Rev. Neurosci. 2006, 7(9), 710-23; Lansbury et al., Nature. 2006, 443(7113), 774-9; Rubinsztein et al., Nature 2006, 443(7113), 780-6).

Methods of Using Butaclamol in Accordance with the Present Invention

The present invention encompasses the recognition that butaclamol can be effective in treating patients with amyotrophic lateral sclerosis (ALS) or other neurodegenerative diseases characterized by the presence of aberrant protein aggregates. Without wishing to be bound by any particular theory or mechanism of action, methods of the invention are useful in inhibiting or reversing abnormal protein aggregation or reducing the toxicity of protein aggregation (e.g., SOD1 or TDP-43). The invention provides methods for treating a subject suffering from or susceptible to ALS or other neurodegenerative disease including the step of administering to the subject a therapeutically effective amount of butaclamol or a pharmaceutical composition thereof. In certain embodiments, the subject is a transgenic mouse. In certain embodiments, the subject is an adult human. In certain embodiments, the ALS being treated is familial ALS. In certain embodiments, the ALS being treated is sporadic ALS.

In some embodiments, the neurodegenerative disease characterized by the presence of aberrant protein aggregates is Parkinson's disease (PD), diffuse Lewy body disease (DLBD), multiple system atrophy (MSA), and pantothenate kinase-associated neurodegeneration (PANK), Huntington's Disease (HD), prion diseases (e.g., Creutzfeldt Jakob disease), Alzheimer's Disease (AD), or frontotemporal lobar degeneration.

In some embodiments, the invention provides a method comprising steps of administering to a subject suffering from or susceptible to ALS an effective amount of butaclamol, such that the severity or incidence of one or more symptoms of ALS is reduced, or its onset is delayed. In some embodiments, butaclamol is administered in the form of a salt or pharmaceutically acceptable composition thereof. In certain embodiments, butaclamol is administered in accordance with the present invention to subjects suffering from or susceptible to a neurodegenerative disease, disorder, or condition in a form or composition and/or according to a regimen established as useful in the treatment of schizophrenia as discussed above. In certain embodiments, the subject suffering from or susceptible to ALS is a human from about 40 to about 85 years of age.

In some embodiments, the ALS being treated is characterized by the presence of abnormal protein aggregates such as, for example, SOD1 protein aggregates or TDP-43 protein aggregates. Exemplary such SOD1 protein aggregates include G93A SOD1 and G85R SOD1 protein aggregates. Without wishing to be bound by any particular theory, use of butaclamol in the treatment of ALS may reduce or delay the formation of such protein aggregates.

In some embodiments, butaclamol is administered once a day. In some embodiments, butaclamol is administered two, three, four, or five times a day. In some embodiments, butaclamol is administered every other day. In some embodiments, butaclamol is administered every two days. In some embodiments, butaclamol is administered every three days. In some embodiments, butaclamol is administered every four days. In some embodiments, butaclamol is administered every five days. In some embodiments, butaclamol is administered every six days. In some embodiments, butaclamol is administered once a week. In some embodiments, butaclamol is administered at intervals as instructed by a physician for the duration of the life of the subject being treated. In certain embodiments, butaclamol is administered as many times a day as necessary to provide a therapeutically effective amount of butaclamol to treat a subject suffering from or susceptible to ALS.

In some embodiments, the subject suffering from or susceptible to ALS is a mammal. In some embodiments, the subject suffering from or susceptible to ALS is a rodent, such as a rat or mouse, for example, a mouse model of ALS. In certain embodiments, the subject suffering from or susceptible to ALS is a human. In certain embodiments, the human is about 20, 30, 40, 50, 60, 70, 80, 90, or 100 years of age. In certain embodiments, the human is between 40 and 85 years of age.

The efficacy of butaclamol in the treatment of neurodegenerative diseases according to the present invention may be evaluated and followed using any method known in the medical arts. The treatment of ALS may be evaluated, for example, by physical examination, laboratory testing, imaging studies, electrophysiological studies, etc. In some embodiments, the treatment of ALS may be evaluated by monitoring the subject being treated. In some embodiments, the subject is monitored by monitoring motor function. In some embodiments, the subject is monitored by monitoring body weight. In some embodiments, the subject is monitored by monitoring survival time. In some embodiments, the subject is monitored one, two, three, four, or five times a day. In some embodiments, the subject is monitored one, two, three, four or five times a week. In some embodiments, the subject is monitored twice a week. In some embodiments, monitoring is continuous. In some embodiments, monitoring occurs for the duration of the subject's life. In certain embodiments, the subject is monitored one, two, or three times a day by monitoring body weight. In some embodiments, the subject is a human and is monitored using any of the methods known in the medical arts suitable for monitoring humans suffering from or susceptible to a neurodegenerative disease such as ALS. Exemplary such methods of monitoring include monitoring neurological function, respiratory function (e.g., pulmonary function test), muscle strength, speech, swallowing function, etc. In some embodiments, monitoring may comprise checking for signs of toxicity; in certain embodiments, toxicity is measured using any of the methods previously developed to measure toxicity of butaclamol in patients being treated for schizophrenia.

As noted above, in some embodiments, butaclamol used in accordance with the present invention comprises (+) butaclamol. In certain embodiments, the butaclamol is about 50% (+) butaclamol. In certain embodiments, the butaclamol is about 60% (+) butaclamol. In certain embodiments, the butaclamol is about 70% (+) butaclamol. In certain embodiments, the butaclamol is about 80% (+) butaclamol. In certain embodiments, the butaclamol is about 90% (+) butaclamol. In certain embodiments, the butaclamol is about 95% (+) butaclamol. In certain embodiments, the butaclamol is about 99% (+) butaclamol. In some embodiments, substantially all of the butaclamol so contained and/or delivered is (+) butaclamol. In some embodiments, at least 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% or more of the butaclamol so contained and/or delivered is (+) butaclamol.

Dosages of butaclamol utilized in accordance with the present invention may vary with the form of administration and/or with the particular subject being treated for ALS. In general, butaclamol is most desirably administered at a concentration level that will afford effective results without causing any harmful or deleterious side-effects (e.g., extrapyramidal effects). In some embodiments, butaclamol is administered in doses ranging from about 0.5 to about 500 mg/kg/day. In some embodiments, butaclamol is administered in doses ranging from about 5 to about 100 mg/kg/day. In some embodiments, butaclamol is administered in doses ranging from about 10 to about 100 mg/kg/day. In some embodiments, butaclamol is administered in doses ranging from about 20 to about 100 mg/kg/day. In some embodiments, butaclamol is administered in doses ranging from about 30 to about 100 mg/kg/day. In some embodiments, butaclamol is administered in doses ranging from about 40 to about 100 mg/kg/day. In some embodiments, butaclamol is administered in doses ranging from about 50 to about 100 mg/kg/day. In some embodiments, butaclamol is administered in doses ranging from about 60 to about 100 mg/kg/day. In some embodiments, butaclamol is administered in doses ranging from about 70 to about 100 mg/kg/day. In some embodiments, butaclamol is administered in doses ranging from about 80 to about 100 mg/kg/day. In some embodiments, butaclamol is administered in doses ranging from about 90 to about 100 mg/kg/day. In some embodiments, butaclamol is administered in doses of less than about 20 mg/day. In some embodiments, butaclamol is administered in doses ranging from about 1 mg/kg/day to about 50 mg/kg/day. In some embodiments, butaclamol is administered in doses ranging from about 1 mg/kg/day to about 40 mg/kg/day. In some embodiments, butaclamol is administered in doses ranging from about 1 mg/kg to about 30 mg/kg/day. In some embodiments, butaclamol is administered in doses ranging from about 1 mg/kg/day to about 20 mg/kg/day. In some embodiments, butaclamol is administered in doses ranging from about 1 mg/kg/day to about 10 mg/kg/day. In some embodiments, butaclamol is administered in doses ranging from about 10 mg/kg/day to about 50 mg/kg/day. In some embodiments, butaclamol is administered in doses ranging from about 10 mg/kg/day to about 40 mg/k/day. In some embodiments, butaclamol is administered in doses ranging from about 10 mg/kg/day to about 30 mg/kg/day. In some embodiments, butaclamol is administered in doses ranging from about 10 mg/kg/day to about 20 mg/kg/day. In some embodiments, butaclamol is administered in doses ranging from about 20 mg/kg/day to about 50 mg/kg/day. In some embodiments, butaclamol is administered in doses ranging from about 20 mg/kg/day to about 40 mg/kg/day. In some embodiments, butaclamol is administered in doses ranging from about 20 mg/kg/day to about 30 mg/kg/day. In some embodiments, butaclamol is administered in doses ranging about 25 mg/kg/day to about 50 mg/kg/day. In some embodiments, butaclamol is administered in doses ranging about 30 mg/kg/day to about 50 mg/kg/day. In some embodiments, butaclamol is administered in doses ranging from about 30 mg/kg/day to about 40 mg/kg/day. In some embodiments, butaclamol is administered in doses ranging from about 40 mg/kg/day to about 50 mg/kg/day. In some embodiments, butaclamol is administered in doses less than about 10 mg/kg/day. In some embodiments, butaclamol is administered in doses less than about 5 mg/kg/day. In some embodiments, butaclamol is administered in doses less than about 2 mg/kg/day. In some embodiments, butaclamol is administered in doses less than about 1 mg/kg/day. In some embodiments, butaclamol is administered in doses less than about 0.1 mg/kg/day. In some embodiments, butaclamol is administered in doses less than about 0.01 mg/kg/day. In some embodiments, butaclamol is administered in doses less than about 0.001 mg/kg/day. In some embodiments, butaclamol is administered in doses of about 0.1, 0.2, 0.3, 0.4, 0.5, 0.6, 0.7, 0.8, 0.9, or 1.0 mg/kg/day. In some embodiments, butaclamol is administered in doses of about 0.01, 0.02, 0.03, 0.04, 0.05, 0.06, 0.07, 0.08, or 0.09 mg/kg/day. In some embodiments, butaclamol is administered in doses ranging from about 0.1 mg/kg/day to about 1 mg/kg/day. In some embodiments, butaclamol is administered in doses ranging from about 0.01 mg/kg/day to about 0.1 mg/kg/day. In some embodiments, butaclamol is administered in doses ranging from about 0.001 mg/kg/day to about 0.01 mg/kg/day. In some embodiments, butaclamol is administered to a subject in doses that do not produce extrapyramidal effects in the subject. In some embodiments, doses described above used in the treatment of schizophrenia are in accordance with the present invention. In some embodiments, butaclamol is administered to a subject in doses lower than those used in the treatment schizophrenia.

In some embodiments, butaclamol is administered systemically in any one of the doses described herein and suitable for the treatment of ALS. Systemic administration may comprise enteral or parenteral administration. In certain embodiments, systemic administration comprises oral administration in solid or solution form in any one of the doses described herein. In certain embodiments, butaclamol is administered parenterally in any one of the doses described herein. In certain embodiments, butaclamol is administered intraperitoneally in any one of the doses described herein and the subject is a mouse or rat with ALS. In some embodiments, butaclamol is administered orally and the subject is a human with ALS.

In some embodiments, the invention provides a method comprising steps of administering to a subject suffering from or susceptible to abnormal protein aggregation an amount of butaclamol sufficient to reduce or delay such abnormal protein aggregation. In certain embodiments, reduction or inhibition of abnormal protein aggregation occurs in vivo in a subject with ALS or another neurodegenerative disease characterized by aberrant protein aggregation (e.g., Huntington's disease, prion disease, Alzheimer's disease, or frontotemporal lobar degeneration). In some embodiments, the subject is a mammal. In some embodiments, the subject is a mouse or rat. In some embodiments, the subject is a human. In certain embodiments, the human is about 20, 30, 40, 50, 60, 70, 80, 90, or 100 years of age. In some embodiments, abnormal protein aggregation comprises SOD1 protein aggregates. In certain embodiments, abnormal protein aggregation comprises G93A SOD1 protein aggregates. In certain embodiments, abnormal protein aggregation comprises G85R SOD1 protein aggregates. In certain embodiments, abnormal protein aggregation comprises TDP-43 protein aggregates.

In some embodiments, butaclamol is administered to a subject with a neurodegenerative disease using any method of administration known in the medical arts. In certain embodiments, butaclamol may be administered orally. In certain embodiments, butaclamol may be administered parenterally. In certain embodiments, butaclamol is administered intraperitoneally.

In some embodiments, the invention provides a method comprising the steps of administering to a cell in vitro an amount of butaclamol effective to inhibit or reverse the toxic effect of abnormal protein aggregation. In certain embodiments, contact occurs in vitro, and the cell is derived from a mammalian cell line. In certain embodiments, contact occurs in vitro, and the cell is derived from a PC12 cell line. In certain embodiments, PC12 cells may additionally contain a detectable moiety to measure the extent of inhibition of aggregation. In certain embodiments, a detectable moiety is associated with a protein (e.g., a type of SOD1 protein, such as G93A SOD1 and/or G85R SOD1, or a type of TDP-43 protein). In certain embodiments, the detectable moiety is a fluorescent moiety (e.g., a YFP tag). In some embodiments, the detectable moiety is a phosphorescent moiety, a radiolabel, or any other detectable moiety known in the art, and may be detected using any of the methods known in the art. In some embodiments, the detectable moiety may be detected using a high content microscopy system to allow for high-throughput screening. In certain embodiments, the detectable moiety allows for the measurement of cell viability.

In some embodiments, the invention provides a method comprising the steps of administering to a cell in vitro an amount of butaclamol effective to protect against aggregated SOD1. In certain embodiments, protection occurs in vitro in a cell culture. In some embodiments, compounds of the invention are contacted with a cell line in vitro and the cell line is a mammalian cell line. In certain embodiments, the cell line is the PC12 cell line. In some embodiments, cells are associate with a detectable moiety such as those described above. In some embodiments, cells contain a protein labeled with a detectable moiety. In certain embodiments, the protein is SOD1 (e.g., G93A SOD1 and/or G85R SOD1) and the detectable moiety is a fluorescent moiety. In certain embodiments, the detectable moiety is a fluorescent moiety (e.g., a YFP tag) that may be detected using a high content microscopy system to allow for high-throughput screening. In some embodiments, the detectable moiety is a phosphorescent moiety, an epitope, radiolabel, or any other detectable moiety known in the art, and may be detected using any of the methods known in the art. In certain embodiments, the detectable moiety allows for the measurement of cell viability.

In some embodiments, the invention provides a method comprising the steps of administering to a cell in vitro an amount of butaclamol effective to modulate proteasome function. In certain embodiments, the cell is derived from a mammalian cell line. In some embodiments, the cell is derived from a PC12 cell line or a HeLa cell line. In certain embodiments, the cells contain a detectable moiety to measure the extent to which proteasome activity is inhibited. In certain embodiments, the protein is SOD1 (e.g., G93A SOD1 and/or G85R SOD1) and the detectable moiety is a fluorescent moiety. In certain embodiments, the protein is TDP-43. In certain embodiments, the detectable moiety is a fluorescent moiety such as a Ubi-YFP tag. In some embodiments, the detectable moiety is a Ubi-YFP tag and is detectable by fluorescence microscopy. In some embodiments, the detectable moiety is a phosphorescent moiety, an epitope, a radiolabel, or any other detectable moiety known in the art, and may be detected using any of the methods known in the art. In some embodiments, the detectable moiety may be detected using a high content microscopy system to allow for high-throughput screening. In certain embodiments, cell viability is measured.

In some embodiments, the present invention provides systems, methods, and/or reagents to characterize butaclamol compounds and compositions. In some embodiments, the present invention provides assays to identify forms of butaclamol compounds and compositions that protect against protein aggregate-induced cytotoxicity. In certain embodiments, the assays are cell protection assays. Cell protection assays may used to identify butaclamol compounds and compositions that protect cells from the cytotoxic effects of aberrant protein aggregation. In some embodiments, the assays are protein aggregation inhibition assays that are used to identify butaclamol compounds and compositions that inhibit protein aggregation in a cell or in vitro.

In some embodiments, the present invention provides a method of identifying butaclamol compounds and compositions that protect against protein aggregate-induced cytotoxicity comprising contacting a cell expressing SOD1, TDP-43, or another protein susceptible to aggregation with a test compound, incubating the cell with the test compound under suitable conditions for an amount of time sufficient to observe a protective effect against protein aggregate-induced cytotoxicity, and then measuring viability in the cells treated with the test compound. In some embodiments, the extent of protein aggregation-induced cytotoxicity is measured by determining the level of a detectable moiety (e.g., a fluorescent moiety) in the cell.

In certain embodiments, the expressed protein in the cell used in the assay is a mutant SOD1 protein. In certain embodiments, the expressed protein the cell used in the assay is a mutant TDP-43 protein. In some embodiments, the expressed protein is SOD1 protein associated with a detectable moiety. In certain embodiments, the expressed protein is a fluorescently tagged mutant SOD1 protein, and the fluorescent moiety is a YFP tag. In some embodiments, the detectable moiety is a phosphorescent moiety, epitope, or radiolabel. In some embodiments, the detectable moiety is any suitable detectable moiety known to those or ordinary skill in the art and may be detected using any method known in the art. In some embodiments, the detectable moiety is a fluorescent tag (e.g., a YFP tag) that can be detected with a high content microscopy system. In some embodiments, the high content microscopy system detects cell viability and facilitates high-throughput screening of a plurality of butaclamol compounds.

Cells may be pre-treated with an agent that modulates the expression of a protein of interest (e.g., SOD1, TDP-43) in the assay. The agent may, for instance, induce the expression of a gene responsible for the protein of interest (e.g., doxycycline-inducible promoter). In some embodiments, cells may also be treated with an agent that modulates proteasome activity. In certain embodiments, the agent may be a proteasome inhibitor (e.g., MG132). In some embodiments, cell viability of cells pre-treated with an agent described herein is measured using methods described above.

In certain embodiments, the time of incubation of a cell with a butaclamol compound or composition ranges from approximately 1 minute to approximately 1 week. In some embodiments, the time of incubation ranges from approximately 5 minutes to approximately 1 week. In some embodiments, the time of incubation ranges from approximately 30 minutes to approximately 2 days. In some embodiments, the time of incubation ranges from approximately 30 minutes to approximately 1 day. In some embodiments, the time of incubation ranges from approximately 1 hour to approximately 1 day. In some embodiments, the time of incubation ranges from approximately 1 hour to approximately 18 hours. In some embodiments, the time of incubation ranges from approximately 1 hour to approximately 12 hours. In some embodiments, the time of incubation ranges from approximately 1 hour to approximately 6 hours. In some embodiments, the time of incubation ranges from approximately 1 hour to approximately 3 hours. In some embodiments, the time of incubation is approximately 6 hours. In some embodiments, the time of incubation is approximately 12 hours. In some embodiments, the time of incubation is approximately 18 hours. In some embodiments, the time of incubation is approximately 24 hours.

In certain embodiments, the temperature during incubation of a cell with a butaclamol compound or composition ranges from approximately 20° C. to approximately 45° C. In certain embodiments, the temperature ranges from approximately 20° C. to approximately 40° C. In certain embodiments, the temperature ranges from approximately 25° C. to approximately 40° C. In certain embodiments, the temperature ranges from approximately 30° C. to approximately 40° C. In certain embodiments, the temperature is approximately 30° C. In certain embodiments, the temperature is approximately 37° C.

Butaclamol compounds or compositions that are active in the above-mentioned assay could theoretically protect against abnormal protein aggregate-induced cytotoxicity through a number of biological mechanisms. The present invention additionally provides methods to screen for butaclamol compounds or compositions that protect against abnormal protein-aggregate induced cytotoxicity wherein the protein aggregation is inhibited in a non-specific manner.

Butaclamol compounds or compositions which inhibit aberrant protein aggregation can be identified using methods similar to those described above in the aforementioned cytotoxicity assay. In some embodiments, the present invention provides a method of identifying butaclamol compounds or compositions that inhibit aberrant protein aggregation comprising contacting a cell expressing SOD1 or other protein susceptible to aggregation with a butaclamol compound or composition, incubating the cell with the compound or composition under suitable conditions, and then measuring the extent of protein aggregation in the cells treated with the butaclamol compound or composition as compared to a control. In certain embodiments, the extent of inhibition of protein aggregation is measured by staining the protein aggregates with a detectable stain (e.g., Image-iT plasma membrane dye). In some embodiments, the detectable stain is detected using a scanning device (e.g., Cellomics Arrayscan). In certain embodiments, the protein aggregates are detected using any method of detecting protein aggregates known in the art.

Butaclamol compounds and compositions identified using the above-mentioned assays may be further examined using biological assays to guide structure-activity relationship (SAR) analyses of the identified compounds. Biological assays and SAR analyses are known to those of skill in the art.

### Pharmaceutical Compositions

In some embodiments, the present invention provides pharmaceutical compositions, which comprise a therapeutically effective amount of butaclamol, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In another aspect, the present invention provides “pharmaceutically acceptable” compositions, which comprise a therapeutically effective amount of one or more of the compounds described herein, formulated together with one or more pharmaceutically acceptable carriers (additives) and/or diluents. As described in detail, the pharmaceutical compositions of the present invention may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream or foam; sublingually; ocularly; transdermally; or nasally, pulmonary and to other mucosal surfaces.

The phrase “pharmaceutically acceptable carrier” as used herein means a pharmaceutically acceptable material, composition or vehicle, such as a liquid or solid filler, diluent, excipient, or solvent encapsulating material, involved in carrying or transporting the subject compound from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

In some embodiments, butaclamol for use in accordance with the present invention is provided in a salt form. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include salts such as the sulfate, phosphate, lactate, tartrate, maleate, citrate, hydrochloride (Bruderlein et al., U.S. Pat. No. 3,657,250) and the like. See also, for example, Berge et al. (1977) “Pharmaceutical Salts”, J. Pharm. Sci. 66:1-19; incorporated herein by reference.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of butaclamol which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of butaclamols of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compound, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of butaclamol include powders, sprays, ointments, pastes, creams, lotions, gels, solutions, patches and inhalants. The butaclamol may be mixed under sterile conditions with a pharmaceutically-acceptable carrier, and with any preservatives, buffers, or propellants which may be required.

The ointments, pastes, creams and gels may contain, in addition to butaclamol, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to butaclamol, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches may have the added advantage of providing controlled delivery of butaclamol to the body. Dissolving or dispersing butaclamol in the proper medium can make such dosage forms. Absorption enhancers can also be used to increase the flux of butaclamol across the skin. Either providing a rate controlling membrane or dispersing butaclamol in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise butaclamol in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the butaclamol may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, butaclamol or pharmaceutical preparation is administered orally. In other embodiments, butaclamol or pharmaceutical preparation is administered intravenously. Alternative routs of administration include sublingual, intramuscular, and transdermal administrations.

When butaclamol is administered as a pharmaceutical, to humans and animals, it can be given per se or as a pharmaceutical composition containing, for example, 0.1% to 99.5% (more preferably, 0.5% to 90%) of active ingredient in combination with a pharmaceutically acceptable carrier.

The preparations of the present invention may be given orally, parenterally, topically, or rectally. Butaclamol is of course given in forms suitable for each administration route. For example, it may be administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The phrases “systemic administration,” “administered systemically,” “peripheral administration,” and “administered peripherally” as used herein mean the administration of a compound, drug or other material other than directly into the central nervous system, such that it enters the patient's system and, thus, is subject to metabolism and other like processes, for example, subcutaneous administration.

Butaclamol may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, butaclamol, which may be used in a suitable hydrated form, and/or the pharmaceutical compositions of the present invention, is formulated into pharmaceutically-acceptable dosage forms by conventional methods known to those of skill in the art.

Actual dosage levels of the active ingredients in the pharmaceutical compositions of butaclamol may be varied so as to obtain an amount of the active ingredient that is effective to achieve the desired therapeutic response for a particular patient, composition, and mode of administration, without being toxic to the patient.

The selected dosage level will depend upon a variety of factors including the particular salt or isomer composition of butaclamol used, the route of administration, the time of administration, the rate of excretion or metabolism of the particular composition being employed, the duration of the treatment, other drugs, compounds and/or materials used in combination with butaclamol, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of butaclamol employed in the pharmaceutical composition at levels lower than that required to achieve the desired therapeutic effect and then gradually increasing the dosage until the desired effect is achieved.

In some embodiments, butaclamol or a pharmaceutical composition thereof is provided to a synucleinopathic subject (e.g., a subject with ALS) chronically. Chronic treatments include any form of repeated administration for an extended period of time, such as repeated administrations for one or more months, between a month and a year, one or more years, or longer. In many embodiments, a chronic treatment involves administering butaclamol or a pharmaceutical composition thereof repeatedly over the life of the subject. Preferred chronic treatments involve regular administrations, for example one or more times a day, one or more times a week, or one or more times a month. In general, a suitable dose such as a daily dose of butaclamol will be that amount of butaclamol that is the lowest dose effective to produce a therapeutic effect. Such an effective dose will generally depend upon the factors described above. Generally doses of butaclamol for a patient, when used for the indicated effects, will range from about 0.0001 to about 100 mg per kg of body weight per day. In some embodiments, the daily dosage will range from about 1 to about 50 mg of compound per kg of body weight. In certain embodiments, the daily dosage will range from about 25 to about 50 mg of compound per kg of body weight. However, lower or higher doses can be used. In some embodiments, the dose administered to a subject may be modified as the physiology of the subject changes due to age, disease progression, weight, or other factors.

If desired, the effective daily dose of the active compound may be administered as two, three, four, five, six, or more sub-doses administered separately at appropriate intervals throughout the day, optionally, in unit dosage forms.

While it is possible for butaclamol to be administered alone, it is preferable to administer it as a pharmaceutical formulation (composition) as described above.

Butaclamol may be formulated for administration in any convenient way for use in human or veterinary medicine, by analogy with other pharmaceuticals.

According to the invention, butaclamol can be formulated or administered using methods that help the it cross the blood-brain barrier (BBB) to a greater extent than would occur without a particular formulation of route of administration. The vertebrate brain (and CNS) has a unique capillary system unlike that in any other organ in the body. The unique capillary system has morphologic characteristics which make up the blood-brain barrier (BBB). The blood-brain barrier acts as a system-wide cellular membrane that separates the brain interstitial space from the blood. The unique morphologic characteristics of the brain capillaries that make up the BBB are: (a) epithelial-like high resistance tight junctions which literally cement all endothelia of brain capillaries together, and (b) scanty pinocytosis or transendothelial channels, which are abundant in endothelia of peripheral organs.

Antibodies are another method for delivery of compositions of the invention. For example, an antibody that is reactive with a transferrin receptor present on a brain capillary endothelial cell, can be conjugated to a neuropharmaceutical agent to produce an antibody-neuropharmaceutical agent conjugate (U.S. Pat. No. 5,004,697, incorporated herein in its entirety by reference). The method is conducted under conditions whereby the antibody binds to the transferrin receptor on the brain capillary endothelial cell and the neuropharmaceutical agent is transferred across the blood brain barrier in a pharmaceutically active form. The uptake or transport of antibodies into the brain can also be greatly increased by cationizing the antibodies to form cationized antibodies having an isoelectric point of between about 8.0 to 11.0 (U.S. Pat. No. 5,527,527, incorporated herein in its entirety by reference).

A ligand-neuropharmaceutical agent fusion protein is another method useful for delivery of compositions to a host (U.S. Pat. No. 5,977,307, incorporated herein in its entirety by reference). The ligand is reactive with a brain capillary endothelial cell receptor. The method is conducted under conditions whereby the ligand binds to the receptor on a brain capillary endothelial cell and the neuropharmaceutical agent is transferred across the blood brain barrier in a pharmaceutically active form. In some embodiments, a ligand-neuropharmaceutical agent fusion protein, which has both ligand binding and neuropharmaceutical characteristics, can be produced as a contiguous protein by using genetic engineering techniques. Gene constructs can be prepared comprising DNA encoding the ligand fused to DNA encoding the protein, polypeptide or peptide to be delivered across the blood brain barrier. The ligand coding sequence and the agent coding sequence are inserted in the expression vectors in a suitable manner for proper expression of the desired fusion protein. The gene fusion is expressed as a contiguous protein molecule containing both a ligand portion and a neuropharmaceutical agent portion).

The permeability of the blood brain barrier can be increased by administering a blood brain barrier agonist, for example bradykinin (U.S. Pat. No. 5,112,596, incorporated herein in its entirety by reference), or polypeptides called receptor mediated permeabilizers (RMP) (U.S. Pat. No. 5,268,164, incorporated herein in its entirety by reference). Exogenous molecules can be administered to the host's bloodstream parenterally by subcutaneous, intravenous or intramuscular injection or by absorption through a bodily tissue, such as the digestive tract, the respiratory system or the skin. The form in which the molecule is administered (e.g., capsule, tablet, solution, emulsion) depends, at least in part, on the route by which it is administered. The administration of the exogenous molecule to the host's bloodstream and the intravenous injection of the agonist of blood-brain barrier permeability can occur simultaneously or sequentially in time. For example, a therapeutic drug can be administered orally in tablet form while the intravenous administration of an agonist of blood-brain barrier permeability is given later (e.g., between 30 minutes later and several hours later). This allows time for the drug to be absorbed in the gastrointestinal tract and taken up by the bloodstream before the agonist is given to increase the permeability of the blood-brain barrier to the drug. On the other hand, an agonist of blood-brain barrier permeability (e.g., bradykinin) can be administered before or at the same time as an intravenous injection of a drug. Thus, the term “co-administration” is used herein to mean that the agonist of blood-brain barrier and the exogenous molecule will be administered at times that will achieve significant concentrations in the blood for producing the simultaneous effects of increasing the permeability of the blood-brain barrier and allowing the maximum passage of the exogenous molecule from the blood to the cells of the central nervous system).

In other embodiments, butaclamol can be formulated as a prodrug with a fatty acid carrier (and optionally with another neuroactive drug). The prodrug is stable in the environment of both the stomach and the bloodstream and may be delivered by ingestion. The prodrug passes readily through the blood brain barrier. The prodrug preferably has a brain penetration index of at least two times the brain penetration index of the drug alone. Once in the central nervous system, the prodrug, which preferably is inactive, is hydrolyzed into the fatty acid carrier and the butaclamol (and optionally another drug). The carrier preferably is a normal component of the central nervous system and is inactive and harmless. Butaclamol, once released from the fatty acid carrier, is active. Preferably, the fatty acid carrier is a partially-saturated straight chain molecule having between about 16 and 26 carbon atoms, and more preferably 20 and 24 carbon atoms. Examples of fatty acid carriers are provided in U.S. U.S. Pat. Nos. 4,939,174; 4,933,324; 5,994,932; 6,107,499; 6,258,836; and 6,407,137, the disclosures of which are incorporated herein by reference in their entirety.).

The administration butaclamol or pharmaceutical compositions thereof may be for either prophylactic or therapeutic purposes. When provided prophylactically, butaclamol is provided in advance of disease symptoms. The prophylactic administration of butaclamol serves to prevent or reduce the rate of onset of symptoms of ALS. When provided therapeutically, the butaclamol is provided at (or shortly after) the onset of the appearance of symptoms of actual disease. In some embodiments, the therapeutic administration of butaclamol serves to reduce the severity and duration of the disease.

The function and advantage of these and other embodiments of the present invention will be more fully understood from the examples described below. The following examples are intended to illustrate the benefits of the present invention, but do not exemplify the full scope of the invention.

### EXAMPLES

The foregoing has been a description of certain non-limiting embodiments of the invention. Accordingly, it is to be understood that the embodiments of the invention herein described are merely illustrative of the application of the principles of the invention. Reference herein to details of the illustrated embodiments is not intended to limit the scope of the claims.

### Example 1

### Assays for Identification of Compounds that Protect Against Mutant SOD1—Induced Cytotoxicity

### High Throughput Assays

Cultured cells are utilized to conduct high throughput assays for compounds that protect against mutant SOD1-induced cytotoxicity. Two assays are used: first, in the cytotoxicity protection assay, compounds are screened for their ability to protect cells from the cytotoxic effects of aggregated mutant SOD1, irrespective of mechanism of drug action. Second, in the protein aggregation assay, compounds are screened for their ability to reduce aggregation of mutant SOD1. The high throughput cytotoxicity protection assay is the primary screen and compounds active in the primary screen (and their analogs) move forward into the secondary screen for protein aggregation.

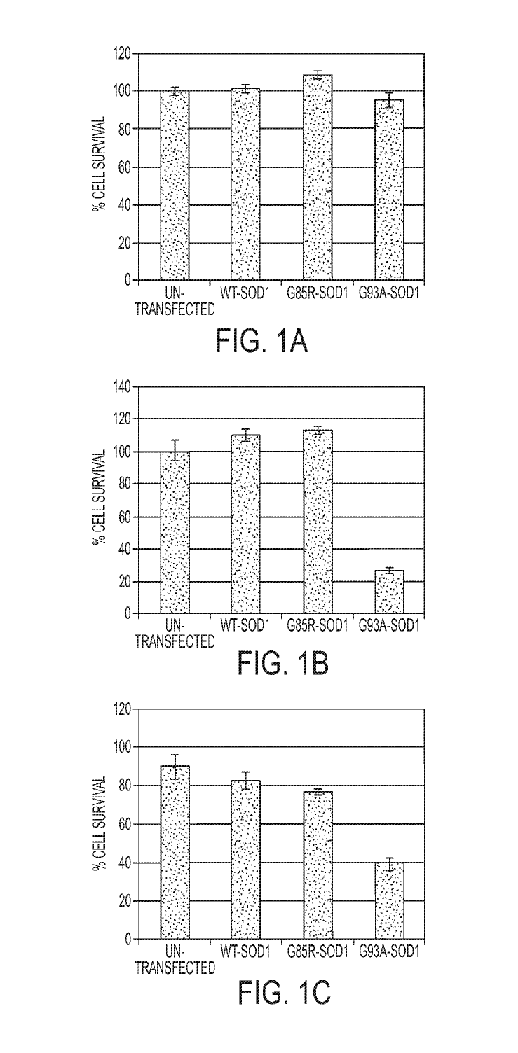

The high throughput cytotoxicity protection assay was carried out in PC12 cells that express mutant G93A SOD1 as a YFP fusion protein from a doxycycline-inducible promoter (Matsumoto et al., J. Cell. Biol. 2005, 171, 75). Several lines of evidence suggest that cytotoxicity of protein aggregates in ALS is due at least in part to inhibition of the proteasome (Bruijin et al. Annu. Rev. Neurosci. 2004, 27, 723-729; Cleveland et al. Nat. Rev. Neurosci. 2001, 2(11), 806). This idea was tested by examining the sensitivity of PC12 cells to SOD1 aggregates in the presence and absence of proteasome inhibitor MG132. PC12 cells expressing no SOD1, wild type SOD1, G85R SOD1 or G93A SOD1 were grown with or without MG132 (FIG. 1). Cells expressing no SOD1, wild type SOD1 and G85R SOD1 were relatively insensitive to MG132, with an IC₅₀ of approximately 400 nM. In contrast, cells expressing G93A SOD1 were approximately 5-fold more sensitive to MG132 (IC₅₀˜75 nM). In these cells, protein aggregation was detected after 24 h and loss of cell viability was detected at approximately 48 h. Qualitatively similar results were obtained with the structurally distinct proteasome inhibitor bortezomib (Velcade®), suggesting that PC12 cells are indeed susceptible to proteasome inhibition and not some other effect of MG132. The ability of protein aggregates to induce cell death was examined by treating G93A SOD1-expressing cells with MG132 for 24 h, removing the MG132 by washing and assaying cell viability after another 24 h. Because the loss of cell viability was similar following MG132 removal (FIG. 1, part C), it is likely that mutant SOD1 aggregates contribute directly to cytotoxicity in PC12 cells. However, this effect is specific for G93A SOD1 suggesting that this mutant may produce higher levels of a toxic aggregated form of SOD1.

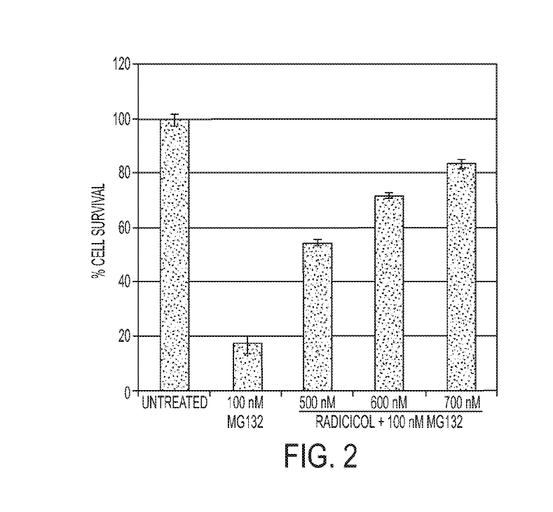

Based on these results, a high throughput screen was developed for compounds that protect against the cytotoxicity of G93A SOD1 protein aggregates using geldanamycin or radicicol as a positive control. PC12 cells expressing G93A SOD1 were treated with 100 nM MG132 with or without co-treatment with geldanamycin or radicicol. The latter compounds inhibit the chaperone HSP90 and induce expression of other chaperones. As anticipated, radicicol reduced formation of protein aggregates and increased cell viability in a dose-dependent manner (FIG. 2). Statistical analysis of the data produced a Z′ value of 0.55, which would predict good performance as a positive control in a high throughput screen (Zhang et al. J. Biomol. Screen 1999, 4(2), 67-73).

### Mutant SOD1 Direct Protein Aggregation Assay.

Compounds that are active in the above assay could theoretically protect against mutant SOD1-induced cytotoxicity through a number of mechanisms, including the following: 1) Compounds could nonspecifically block or reverse protein aggregation via chaperone induction, as observed for radicicol and geldanamycin 2) Compounds could block or reverse the aggregation of a specific aggregated protein form 3) Compounds could interfere with an event downstream of protein aggregation that plays a critical role in mutant SOD1-induced cytotoxicity (e.g., proteasome function). 4) Compounds could act directly on SOD1 in a manner that prevents mutant SOD1 aggregation. These possibilities were tested using an assay that directly measures protein aggregation. In addition, unlike the high throughput cytotoxicity protection assay, the protein aggregation assay is based on G85R SOD1; this broadens the scope of the screening strategy, and should eliminate compounds with highly specific activity (i.e., G93A SOD1 limited) against protein aggregation.

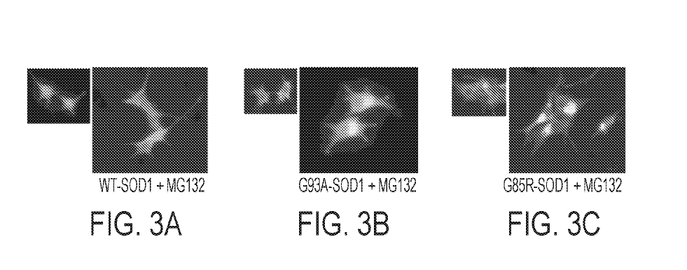

In PC12 cells that express wild-type SOD1, SOD1 was diffusely localized throughout the cell (Matsumoto et al. J. Cell. Biol 2005, 171(1), 75-85). In contrast, G85R SOD1 showed heterogeneous patterns of localization; in most cells, G85R was diffusely localized throughout the cell, but in ˜5% of the cells, G85R SOD1 was localized in large peri-nuclear aggregates. In cells treated with MG132, up to 75% of cells expressing G85R SOD1 contain such protein aggregates (FIG. 3), but no aggregation was observed in cells expressing wild-type SOD1. Cells expressing G93A mutant SOD1 showed an intermediate level of protein aggregation: none of the cells developed protein aggregates in the absence of MG132, and ˜75% of the cells had protein aggregates following treatment with MG132 (FIG. 3). Similar effects were observed in cells treated with bortezomib (Velcade®). Therefore, these effects are likely to be due to MG132-induced proteasome inhibition, and not due to an off-target effect of MG132.

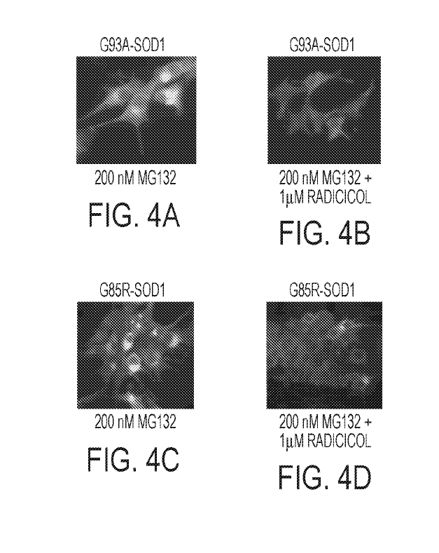

The sensitivity of this assay was optimized by selecting conditions that maximize the difference between active and inactive samples. The identification of a positive control is a crucial step in assay development. Thus, PC12 cells expressing G85R or G93A mutant SOD1 were treated with MG132 to induce protein aggregation, and then co-treated with candidate chemical suppressors of protein aggregation. Two compounds with similar activity were identified in these experiments: geldanamycin and radicicol. Both compounds induce heat shock transcription factor HSF-1, which in turn induces the heat shock response (FIG. 4). Treatment with radicicol reduced the proportion of cells with aggregates from 75% to 25%, a sufficient difference to allow visual scoring for compounds with efficacy equal to or greater than radicicol.

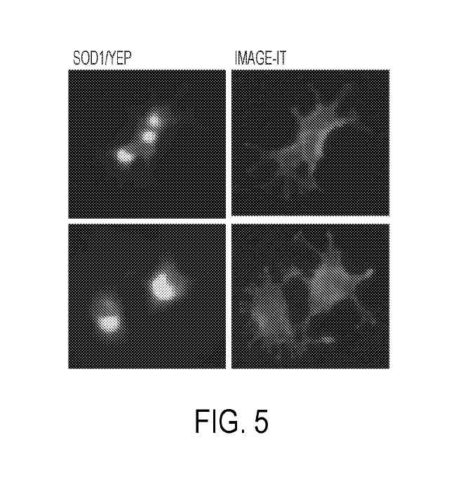

To allow this assay to be used in a high-throughput manner, a Cellomics Arrayscan® high content microscopy system was used for screening and quantification. Initial experiments indicated that G85R SOD1 aggregates were more readily recognized by the high content microscopy system and its computer algorithm. Because the most robust high content assays measure events on a per cell basis, it was necessary to select a fluorescent stain that marks whole cells to be used with a compatible stain that marks intracellular structures. On the basis of pilot experiments with a number of vital dyes, an Image-iT conjugated wheat germ agglutinin (WGA) dye from Molecular Probes was selected and a computer algorithm for detecting WGA was developed. As shown in FIG. 5, WGA provided an excellent cellular marker that did not interfere with detection of YFP-tagged SOD1.

### Example 2

### Identification of Butaclamol

Screening campaign results: The Cambria chemical library is composed of >50,000 small molecule compounds including most FDA approved drugs, a diverse collection of biochemical reagent compounds and structurally diverse random chemistry including compounds unique to Cambria which were sourced via special contractual arrangements. The primary screen yielded 68 primary active compounds, including 67 structurally diverse compounds and a single clinical agent (butaclamol). This collection of actives was then counter-screened to remove artifactual fluorescent compounds and protein synthesis inhibitors that could artifactually block the production of the toxic G93A SOD1 insult. The protection actives were tested for effects on SOD1 protein aggregation and all of these compounds except butaclamol and one structurally novel compound were active in that assay. Chemical structure comparisons of the active compounds grouped them into 17 chemotype classes plus 15 singleton hits.

Neuroleptic therapeutics: Ten neuroleptic therapeutics, representing both the ‘typical’ and ‘atypical’ compound types, were screened in the protection assay (Table 1). Only butaclamol showed activity. Butaclamol was inactive when tested in the aggregation assay. This result suggests that butaclamol is either acting by blocking the toxic effects of the aggregated SOD1 or is blocking the formation of a toxic SOD1 species that is not scored/detected in the aggregation assay.

TABLE 1
Neuroleptic Agent Screening Results

| | | | Activity in |
|---|---|---|---|
| Generic Name | Trade Name | Type | Protection Assay |
| Clozapine | Clozaril | atypical | Inactive |
| Loxapine | Loxitane | typical | Inactive |
| Haloperidol | Haldol | typical | Inactive |
| Chlorprothixene | Taractan | typical | Inactive |
| Trifluoperazine | Stelazine | typical | Inactive |
| Perphenazine | Trilafon | typical | Inactive |
| Butaclamol | | typical | Active |
| Chlorpromazine | Thorazine | typical | Inactive |
| Pimozide | Orap | typical | Inactive |
| Risperidone | Rispedal | atypical | Inactive |

Table 2 presents a side by side comparison of the effects of radicicol (an HSP90 inhibitor chaperone inducer) and butaclamol.

TABLE 2
Comparison of Selected Screening Actives

| | Pro- | | | Cellular |
|---|---|---|---|---|
| | tection | | | Toxicity |
| | ED50 | Protection Efficacy | Aggre- | IC50 |
| Compound | (μM) | % Viability@Conc.(μM) | gation | (μM) |
| Radicicol | ~0.53 | 80% @0.7 | Moderate | ~30 |
| Butaclamol | ~0.84 | 80% @15 | Inactive | <25 |

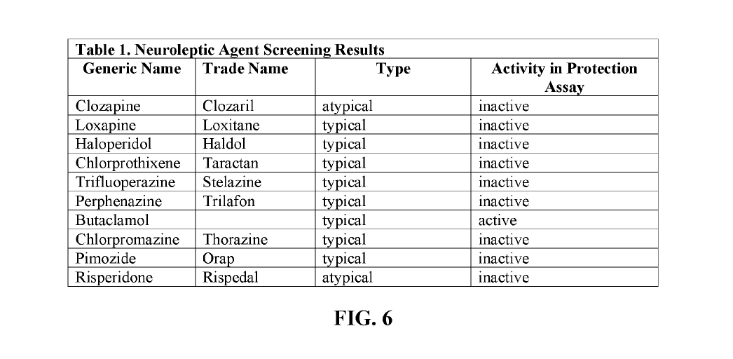

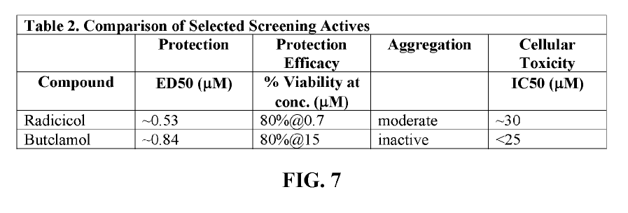

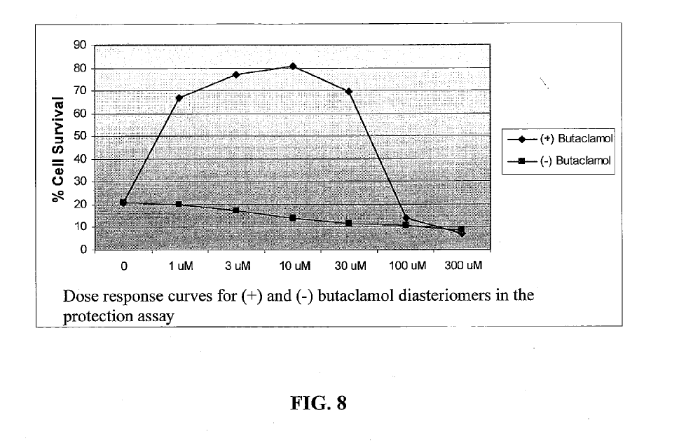

Butaclamol behaves similarly to radicicol in the protection assay, providing incomplete protection. This is likely due to the fact that both of these compounds are toxic at higher doses and that the observed efficacy (maximum viability) is a combination of protective and toxic effects. Butaclamol provides protection over a wide range of concentrations and the plateau of efficacy declines as toxic effects become prominent at higher drug concentrations (FIG. 6). Butaclamol is a diasteriomer and the two different isomeric forms can be physically separated. We therefore tested each separate enantiomer based on the following reasoning: certain NSAID drugs including flurbiprofen will inhibit γ-secretase and lower A1342 levels and thus have potential therapeutic value for the treatment of Alzheimer's disease (Ericson et al., J. Clinical Invest. (2003), 112, 440). Since flurbiprofen is a diasteriomer, one development strategy has been to utilize the flurbiprofen enantiomer lacking cyclooxygenase inhibitory activity as a means to develop treatment that lack toxic gastric side effects. We took an analogous approach with butaclamol and tested the separated enantiomers for protective effects (FIG. 6), in case the distomer lacking dopamine receptor binding and thus extrapyramidal effects (−butaclamol) would be active. In this experiment we found only the eutomer (+butaclamol) showed activity.

### Example 3

### Butaclamol Activity In Vivo

The present example demonstrates that butaclamol has protective effects in the G93A SOD1 ALS mouse model at doses that do not produce extrapyramidal effects. Butaclamol produces extrapyramidal effects in patients at doses as low as 10 mg/day. Therefore dosing in clinical studies should desirably be below this value. Butaclamol has a molecular weight of 362 and assuming a volume of distribution equal to total body water (˜60% of weight), a 10 mg dose could yield a potential maximum concentration of ˜0.65 μM in a 70 kg man. This dose is approximately twice the ED50 for butaclamol in the protection assay, which might suggest that only minimal protective effects is achieved without limiting side effects (i.e., that there might be a narrow therapeutic window). However, the protection assay is a short term protocol that measures effects 48 hours following an acute insult while the SOD1 mouse is a 126 day chronic treatment model. It is possible that lower doses are protective, for example, if administered chronically over a long time period.

Butaclamol was evaluated in the transgenic G93A ALS mouse model (Gurney M. E., et al., Science 264, 1772, 1994). Although butaclamol has been studied in a variety of rodent species as well as in human clinical trials, these experiments were performed approximately 30 years ago and unfortunately the available old data do not provide optimal guidance (Clark et al., Dis. Nerv. Syst. 38, 943, 1977; Nestoros et al., Int. Pharmacopsychiatry 13, 138, 1978; Clark et al., J. Clinical Pharm. 17, 529, 1977; Lippmann et al., Life Sciences 16, 213, 1975; Humber et al., Mol. Pharmacol. 11, 833, 1975; Voith and Herr, Psychopharmacologia 42, 11, 1975). Dose levels reported in these studies ranged from acute doses of 0.1 to 30 mg/kg in a variety of rodent behavioral pharmacology models to doses of 10-50 mg/kg in clinical studies. These doses are used herein as rough guides for the tolerability/dosing/pharmacokinetics experiments below.

### Procedures

Subjects. Animal models of inherited neurological diseases have significantly advanced our understanding of the molecular pathogenesis and hold great promise for developing potential therapeutic strategies for translation to patients. The development of transgenic mice expressing G93A human SOD1 as a mouse model for the human disease has been regarded as a major breakthrough for development of ALS therapeutics. G93A SOD1 transgenic mice develop progressive hind limb weakness, muscle wasting, and neuropathological sequelae similar to those observed in patients with both sporadic and familial ALS. The spinal cord of the G93A SOD1 mouse shows progressive reactive astrogliosis, marked neuronal atrophy, neuronal loss, and the presence of prominent ubiquinated inclusion bodies by 90 days of age. In addition, motor performance deteriorates as the disease progresses. The G93A SOD1 mouse model has played a prominent role in studying disease progression and especially for testing potential therapeutic agents, the latter in part because these animals have a shortened life span of approximately 126 days. In the present study, G93A SOD1 mice and littermate controls are bred from existing colonies at the Bedford VA Medical Hospital. The male G93A SOD1 mice are mated with B6SJL females and the offspring are genotyped by PCR using tail DNA. The number of SOD1 transgenes are assessed by PCR to ensure that transgene copy number remains constant. Mice are housed in micro-isolator cages in complete barrier facilities, and all studies are performed in these facilities. A 12 hour light-dark cycle is maintained and animals are given free access to food and water. Control and transgenic mice of the same age (±2 days) and from the same ‘f generation will be selected from multiple litters to form experimental cohorts (n=20 per group). Standardized criteria for age and parentage are used for placing individual mice into experimental groups/cohorts. Wild type mice are used for initial toxicity, tolerability, and pharmacokinetic studies and ALS mice are used for one-month tolerability studies.

Tolerability, Dosing, and Pharmacokinetics. The tolerable dose range and LD50 for butaclamol was determined in wild type mice by increasing the dose b.i.d. one-fold each injection. The route of administration was via i.p. administration, which was previously reported to produce robust effects in behavioral pharmacology studies, and starting dose range was guided by these prior studies (Lippmann et al., Life Sciences 16, 213, (1975); Humber et al., Mol. Pharmacol. 11, 833, (1975); Voith and Herr, Psychopharmacologia 42, 11, (1975)). One goal was to select a range of doses for the efficacy study starting ten fold below the maximum tolerated dose. Initial pharmacokinetic (pK) studies were conducted by giving animals a single dose, sacrificing them after 30 min, 1 h, 2 h, 4 h, 6 h, and 12 h, and dissecting brains and spinal cords and determining drug concentration in the target tissue. Drug steady-state level was determined in animals that had been dosed for 1 week prior to sacrifice. The range of dosing levels of 0.01, 0.1, and 1 mg/kg once a day were administered throughout the lives of the G93A mice.

Behavioral pharmacology. Behavioral testing for the transgenic G93A SOD1 mice were performed during the light phase of the diurnal cycle since these mice are sufficiently active during that time. Measurements were made for 30 minutes after 10 minutes of acclimation to the box (Opto-Varimex Unit, Columbus Instruments, Columbus, Ohio, USA). Counts of horizontal and vertical motion activity were monitored and quantitative analysis of locomotor activity (resting and ambulatory times), were assessed. The open field box was cleaned before testing each mouse. Each 30 minutes of testing was analyzed as three periods of 10 minute intervals to study the influence of novelty and measured behavior. Mice were coded and investigators were blinded to the genotype and analysis. Testing started on week 4 and performed every other week until the mice could no longer participate.

Efficacy studies. Efficacy was measured using endpoints that clearly indicate neuroprotective function. These include amelioration of degenerative changes in the spinal cord, improved motor function, and prolonged survival. Some mice cohorts were sacrificed at a predetermined time point (120 days) for neuropathological examination, while others were sacrificed at end stage disease using criteria for euthanasia. The latter cohorts were followed temporally for behavioral analyses as well as survival.

Survival. Mice were observed three times daily (morning, noon, and late afternoon) throughout the experiment. Mice were euthanized when disease progression was sufficiently severe that they were unable to initiate movement and right themselves after gentle prodding for 30 seconds.

Body weights. Mice were weighed twice a week at the same time each day. Weight loss is a sensitive measure of disease progression in transgenic G93A SOD1 mice and of toxicity in transgenic and wild type mice.

Motor/behavioral. Quantitative methods of testing motor function are used including Rotarod and analysis of open field behavior. Decline of motor function is a sensitive measure of disease onset and progression. Behavioral testing for the transgenic G93A SOD1 mice were performed during the light phase of the diurnal cycle since these mice are sufficiently active during that time. Measurements were made for 30 minutes after 10 minutes of acclimation to the box (Opto-Varimex Unit, Columbus Instruments, Columbus, Ohio, USA). Counts of horizontal and vertical motion activity were monitored and quantitative analysis of locomotor activity (resting and ambulatory times), were assessed. The open field box was cleaned before testing each mouse. Each 30 minutes of testing was analyzed as three periods of 10 minute intervals to study the influence of novelty and measured behavior. Mice were coded and investigators were blinded to the genotype and analysis. Testing started on week 4 and performed every other week until the mice could no longer participate.

Neuropathology. Selected cohorts (n=10) of treated and untreated G93A SOD1 mice were euthanized at 120 days for isolation and analysis of spinal cord tissue. For this purpose, mice are deeply anesthetized and perfused transcardially with 4% buffered paraformaldehyde at the desired time point. These studies were performed in a blinded manner, to avoid bias in interpretation of the results. Brains were weighed, serially sectioned at 50 pm and stained for quantitative morphology (cresyl violet) to determine gross atrophy and identify ventral neuron loss and astrogliosis. Remaining tissue samples/sections were stored for future use. Stereology was used to quantify gross ventral horn atrophy, neuronal atrophy, and neuronal loss. Remaining tissue samples/sections are stored for prospective mechanistic analyses as necessary.

Analysis. Data sets were generated and analyzed for each clinical and neuropathological measure. Effects on behavior and neuropathology were compared in treatment and control groups. Dose-dependent effects were assessed in each treatment group using multiple two-sided ANOVA tests. Multiple comparisons in the same subject groups were dealt with post hoc. Kaplan-Meier analysis was used for survival and behavioral function.

Neuronal quantitation. Serial lumbar spinal cord tissue sections (n=20) from L3-L5 spinal cord segments were used for gross spinal cord areas and neuronal analysis. Gross areas of the spinal cord sections were quantified from each experimental cohort using NIH Image. From the same sections, the ventral horn was delineated by a line from the central canal laterally and circumscribing the belly of gray matter. Absolute neuronal counts of Nissl-positive neurons were performed in the ventral horns in the lumbar spinal cord. Only those neurons with nuclei were counted. All counts were performed with the experimenter (JM) blinded to treatment conditions. Counts were performed using Image J (NIH) and manually verified and the data represent the average neuronal number from the sections used.

Interpretation. Compound efficacy is evaluated using behavioral and neuropathological endpoints. Results for the test compound are compared with results from compounds with established efficacy and neuroprotective action in the G93A SOD1 mouse model. These experiments directly test whether butaclamol provides therapeutic benefit and, if so, the magnitude of the benefit. Along the way, useful information about solubility, administration, and toxicity are also obtained. Treatment regimens that show efficacy with respect to behavioral and neuropathological outcome measures in the G93A mice at doses not predicted to produce extrapyramidal effects would suggest that butaclamol is a potential clinical lead with the probability for delaying onset or slowing the progression of ALS in humans.

### Results:

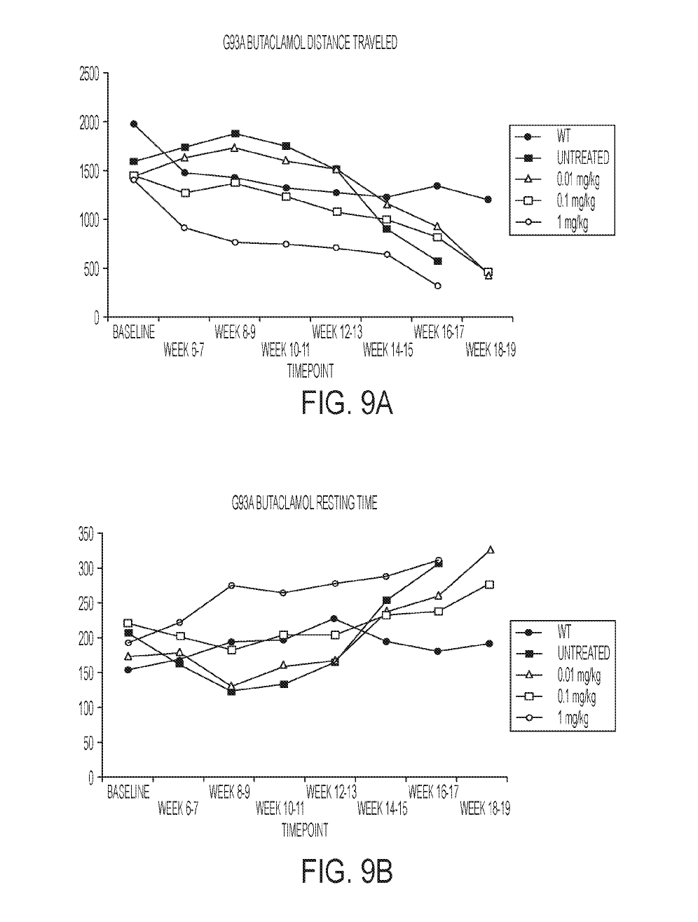

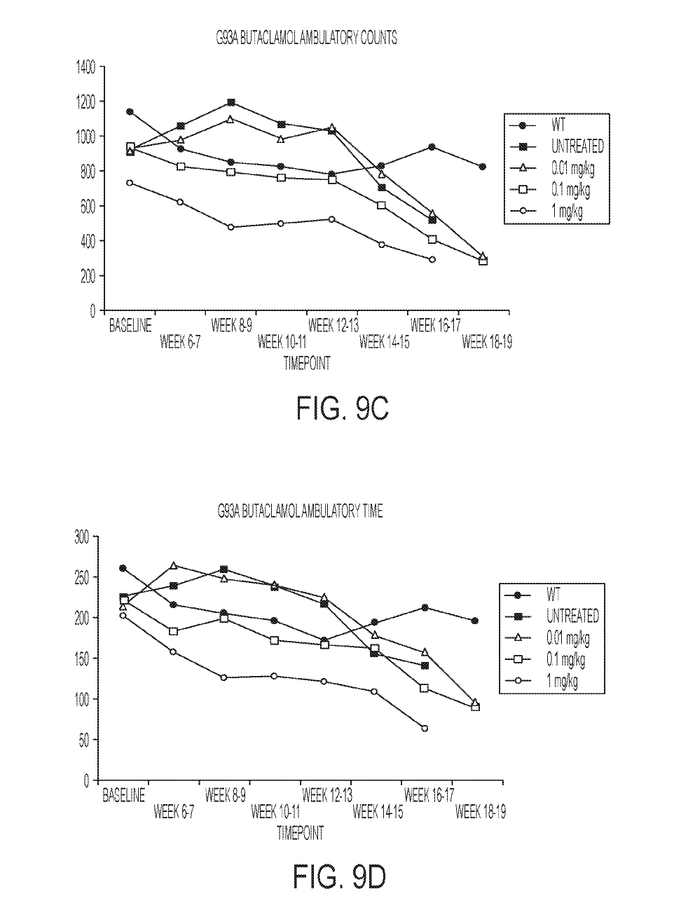

Behavioral results of Open-Field analysis showed marked significant differences in out come measures between wild type littermate mice and untreated G93A mutant mice (FIG. 9). In comparison to wild type mice, there was significant increase in hyperactivity in the untreated G93A mice in distance traveled, ambulatory counts, and ambulatory time starting at 6 weeks through 12-13 weeks. After the 12-13 week time point, there was a significant reduction in distance traveled, ambulatory counts, and ambulatory time in G93A mice, in comparison to the wild type littermate control mice. Resting time was the mirror antithesis of motor movement measures, with a significant reduction from 6 weeks through 12-13 weeks, with increased resting time after 12-13 weeks. In contrast, treated G93A mice showed motor performance changes in butaclamol-treated mice related to dose administration.

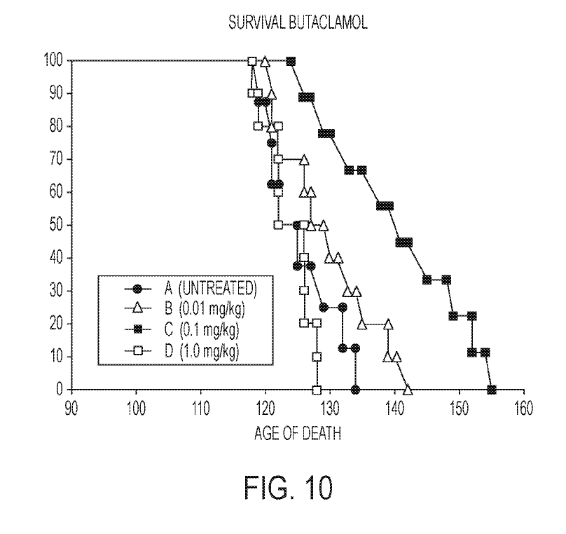

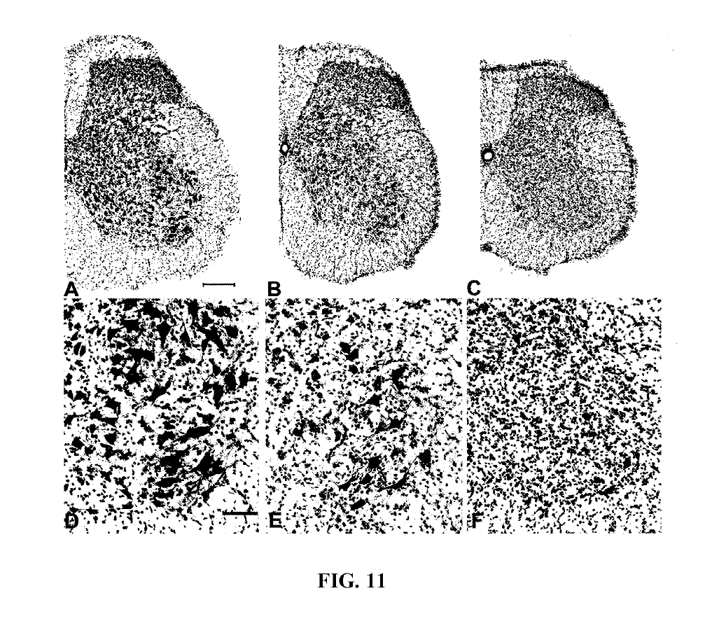

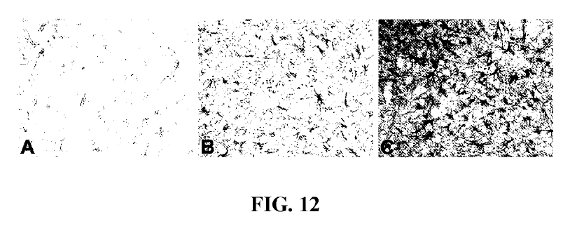

Three different doses (low, medium, and high) of butaclamol were tested. Parameters evaluated include (1) distance traveled; (2) resting time; (3) ambulatory count; and (4) ambulatory time. These results are presented in FIG. 9. Overall survival was also assessed (see FIG. 10), and neuropathological analyses were performed (see FIGS. 11 and 12).

As can be seen with reference to FIG. 9, the low butaclamol dose (0.01 mg/kg) in G93A mice paralleled the untreated G93A mice for distance traveled, resting time, ambulatory count, and ambulatory time over the 6 week to 12-13 week time period. These findings are consistent with a conclusion that the low dose butaclamol treatment had no significant effect over that time period. After 12-13 weeks, mice treated with the low butaclamol dose may have shown some improvement (i.e., behavior trending toward wild type) at least with respect to resting time.

On the other hand, FIG. 9 demonstrates that G93A mice treated with the medium dose of butaclamol showed behaviors not significantly different from wild type mice in the period between 6 weeks and 12-13 weeks. Thus, the medium dose butaclamol treatment appeared to correct these deficits otherwise observed in G93A mice in this time period. Beyond the 12-13 week time point, behavior of G93A mice receiving medium-dose butaclamol diverged from wild type mice, trending toward untreated mice. Specifically, ambulatory distance, ambulatory counts, and ambulatory time decreased, and resting time increased. However, at least some of the parameters (e.g., resting time specifically) remained significantly different from untreated mice.

Mice that received the high butaclamol dose (1 mg/kg) showed significantly reduced motor activity outcome measures throughout treatment, as compared with wild type or untreated G93A mice.

No significant extrapyramidal effects were observed with any of the treated mice. A climbing assay was used to test for extrapyramidal effects.

Butaclamol treatment with low and medium doses extended survival in the G93A ALS mice in a dose dependent manner (FIG. 10). The medium dose extended survival by 12.7% as compared with untreated G93A mice. Average life span was 125.1±3.8 days for untreated G93A mice; 129.3±4.1 days for low dose treated G93A mice; 141.0±5.3 days for medium dose treated G93A mice; and 123.7±6.2 days for high dose treated G93A mice.

Neuropathological analysis at 120 days revealed marked gross spinal cord atrophy, with increased astrogliosis and neuronal loss in the ventral horns from the lumbar spinal cord in untreated G93A mice, in comparison to wild type littermate control mice (FIG. 11). The pathological findings in the untreated G93A mice were significantly reduced by butaclamol (0.1 mg/kg) administration. There was significant gross atrophy of the lumbar spinal cord in untreated G93A mice, in comparison to WT littermate control mice (WT littermate control: 3.18±0.24×10⁶ μm³, untreated G93A mice: 2.25±0.49×10⁶ μm³, P<0.001) with significant amelioration of gross spinal cord atrophy in butaclamol-treated mice at the 0.1 mg·kg dose (butaclamol treated G93A mice: 2.97±0.31×10⁶ μm³, P<0.01) (FIG. 11). The gross atrophy was largely associated with the ventral horn of the lumbar spinal cord (WT littermate control: 7.29±0.41×10⁵ μm³′ untreated G93A mice: 5.28±0.63×10⁵ μm³, P<0.01) and was significantly improved by butaclamol (0.1 mg/kg) treatment (butaclamol-treated G93A mice: 6.55±0.37×10⁵ μm, P<0.05) (FIG. 11 (B)). Consistent with the above findings, there was marked ventral horn neuronal loss in the untreated G93A mice, as compared to the WT littermate control mice (WT littermate control: 98.1±12.9; untreated G93A mice: 22.4±15.8, P<0.0001) (FIG. 11 (D and F)), with significant reduction in neuronal loss as a consequence of butaclamol administration (butaclamol-treated G93A mice: 57.3±18.2, P<0.01) (FIG. 11 (E)). Reactive astrogliosis, a prominent finding in untreated G93A mice spinal cord specimens, was ameliorated by butaclamol treatment, as shown by the marked reduction of glial fibrillary acidic protein expression in the lumbar spinal cord (FIG. 12).

### Significance

Re-purposing a known clinical agent for a new indication is by far the most rapid path to bring a new therapy to patients. However, this approach often has significant limitations. First of all, many compounds are optimized for high selectivity/specificity for their initially intended target and robust off target effects are uncommon. Second, many compounds have a pharmacologically limited route of administration (e.g., topical) that may be desirable for their initial approved use but may bar the application for another disease. This is a particular challenge for a neurodegenerative disease therapeutic, since the vast majority of drugs do not penetrate the blood brain barrier. The demonstration that butaclamol has activity as a potential therapeutic for ALS is particularly provident. Butaclamol is an orally active, blood brain barrier penetrating compound that was designed for long term use. Therefore butacamol has already surmounted many barriers for use as an ALS therapeutic.

### EQUIVALENTS

Having described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements, and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A method comprising steps of:
administering to a subject suffering from or susceptible to amyotrophic lateral sclerosis an effective amount of butaclamol, such that the severity or incidence of one or more symptoms of ALS is reduced, or its onset is delayed.

2. The method of claim 1, wherein the butaclamol is administered in the form of a salt.

3. A method comprising steps of:
administering to a subject suffering from or susceptible to abnormal protein aggregation an amount of butaclamol sufficient to reduce or delay such abnormal protein aggregation.

4. The method of claim 1, wherein the butaclamol comprises (+) butaclamol.

5. The method of claim 1, wherein the butaclamol is about 50% (+) butaclamol.

6. The method of claim 1, wherein the butaclamol is about 60% (+) butaclamol.

7. The method of claim 1, wherein the butaclamol is about 70% (+) butaclamol.

8. The method of claim 1, wherein the butaclamol is about 80% (+) butaclamol.

9. The method of claim 1, wherein the butaclamol is about 90% (+) butaclamol.

10. The method of claim 1, wherein the butaclamol is about 95% (+) butaclamol.

11. The method of claim 1, wherein the butaclamol is about 99% (+) butaclamol.

12. The method of claim 3, wherein the protein aggregates are G93A SOD1 protein aggregates.

13. The method of claim 3, wherein the protein aggregates are G85R SOD1 protein aggregates.

14. The method of claim 1, wherein the subject is a test subject.

15. The method of claim 1, wherein the subject is a human.

16. The method of claim 1, wherein the subject is an adult human.

17. The method of claim 1, wherein the compound is administered in doses of less than about 20 mg/day.

18. The method of claim 1, wherein the compound is administered in doses ranging from about 1 mg/kg/day to about 50 mg/kg/day.

19. The method of claim 1, wherein the compound is administered in doses ranging from about 1 mg/kg/day to about 40 mg/kg/day.

20. The method of claim 1, wherein butaclamol is administered in doses ranging from about 1 mg/kg/day to about 30 mg/kg/day.

21-57. (canceled)
